(19) United States
(12) Patent Application Publication
(10) Pub. No.: US 2025/0199291 A1
(43) Pub. Date: Jun. 19, 2025

(54) ILLUMINATOR END PART

(71) Applicant: Crea IP B.V., Vierpolders (NL)

(72) Inventor: Jan Wijnand Okkerse, Vierpolders (NL)

(73) Assignee: Crea IP B.V., Vierpolders (NL)

(21) Appl. No.: 18/847,267

(22) PCT Filed: Mar. 29, 2023
(86) PCT No.: PCT/NL2023/050161

§ 371 Date: Sep. 16, 2024

(30) Foreign Application Priority Data
Mar. 30, 2022 (NL) 2031446

(51) Int. Cl. G02B 23/24; G02B 1/04

(52) U.S. Cl. CPC G02B 23/2469; G02B 1/04; G02B 23/243

(57) ABSTRACT

Provided herein is an assembly of an ophthalmic illuminator instrument and an illuminator end part. The illuminator instrument has an elongate light guiding end part, extending in a first direction from an instrument side towards a light deflection part side. The illuminator end part has an elongated sleeve part having a bore extending in a first direction with a diameter corresponding to an outside diameter of a light guiding end part of the ophthalmic illuminator instrument and tapering from an instrument side toward a light deflection part, a light deflection part arranged to guide light originating from the light guiding end part in a direction away from the first direction, and an instrument adapter part that is releasably connected to the illuminator instrument.

### FIELD OF THE INVENTION

The present invention relates to an assembly of an ophthalmic illuminator instrument and an illuminator end part.

### BACKGROUND ART

US patent publication U.S. Pat. No. 5,582,608 discloses a lamellar illumination apparatus for eye surgery. A support fixture is provided with a light emitter, and directs light to a surgical field tangentially to the cornea, at an angle of between 0 to 45 degrees to the plane of the eye iris. The light entering the eye travels along the lamellae of the cornea, and as a result very little light reaches the back of the eye or is directed towards a surgical microscope causing glare. The light emitter may be mounted on or incorporated in an eyelid speculum or a fixation ring.

US patent publication US2021/0177393 discloses a trans-scleral illumination system for vitreoretinal surgery, having a speculum with two arms with blades at distal ends thereof. Optical fibers are present running through the arms with one or more end points located within the blades. The position and orientation of the end faces of the fibers determine the direction of light from the illumination system

### SUMMARY OF THE INVENTION

The present invention seeks to provide a simple add-on design feature to existing ophthalmological instruments, such as (endo-) illuminators or laser probes, which allow to re-direct light in a desired direction different from the direction of light provided by the instrument itself.

According to the present invention, an Illuminator end part as defined above is provided, the Illuminator end part comprising an elongated sleeve part having a bore extending in a first direction with a diameter corresponding to an outside diameter of a light guiding end part of the ophthalmic illuminator instrument, and a light deflection part arranged to guide light originating from the light guiding end part in a direction away from the first direction.

### SHORT DESCRIPTION OF DRAWINGS

The present invention will be discussed in more detail below, with reference to the attached drawings, in which

FIG. 1 shows a perspective view of an embodiment of the Illuminator end part according to the present invention;

FIG. 2 shows a cross sectional view of the Illuminator end part shown in FIG. 1 attached to an ophthalmic illuminator instrument;

FIG. 3 shows a detailed cross sectional view of a tip of the Illuminator end part of FIG. 1; and

FIG. 4 shows a view of the combination of Illuminator end part and ophthalmic illuminator instrument during actual use.

### DESCRIPTION OF EMBODIMENTS

The present invention embodiments provide a simple and cost-efficient add-on functionality to ophthalmological instruments, such as (endo-) illuminators, which allow a broader spectrum of use of these instruments by providing a suitable re-direction of light from the instrument.

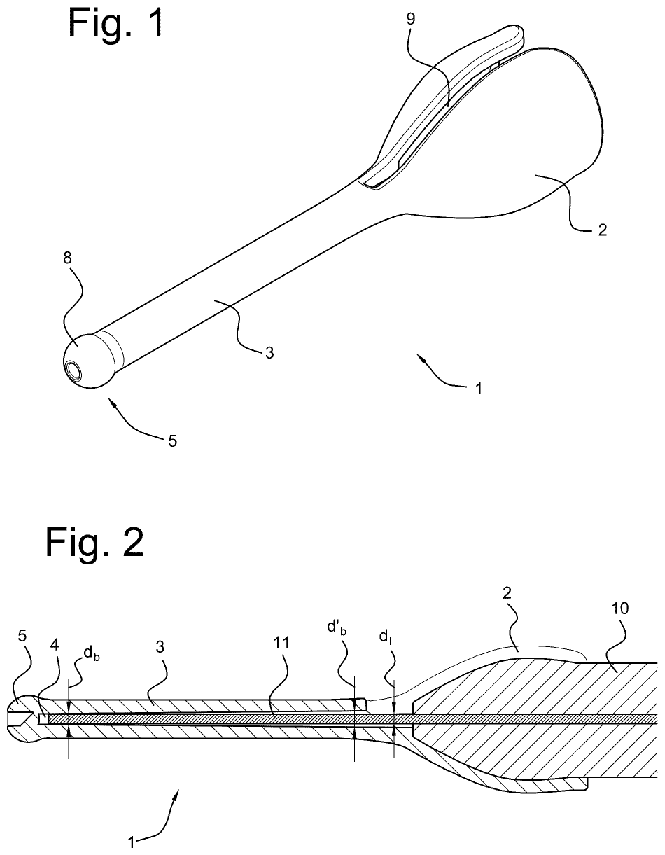

FIG. 1 shows a perspective view of an embodiment of the Illuminator end part 1 according to the present invention, comprising an (optional) instrument adapter part 2 allowing to attach the Illuminator end part 1 to attach to an ophthalmic illuminator instrument 10 (see FIG. 2 below), an elongated sleeve part 3 and a light deflection part 5. In other words, in a first group of embodiments, the Illuminator end part 1 comprises an instrument adaptor part 2, an elongated sleeve part 3 having a bore 4 extending in a first direction (i.e. a. longitudinal direction of the elongated sleeve part 3) with a diameter corresponding to an outside diameter of a light guiding end part 11 of the ophthalmic illuminator instrument 10, and a light deflection part 5 arranged to guide light originating from the light guiding end part 11 in a direction away from the first direction. This allows to have light exiting the combined ophthalmic illuminator instrument 10 and Illuminator end part 1 in a direction different from the regular direction of light of the ophthalmic illuminator instrument 10, i.e. in a longitudinal direction. In general, various directions, ranges and opening angles may be provided for the light exiting the light deflection part 5 of the Illuminator end part 1.

In a further embodiment, as shown in FIG. 1, the light deflection part 5 comprises an outer surface 8 for scleral depression, wherein the outer surface 8 has a maximum outer diameter larger than an outside diameter of the elongated sleeve part 3. The outer surface 8 on the light deflection part 5 provides an additional functionality to the combination of ophthalmic illuminator instrument 10 and Illuminator end part 1, as it is possible for the surgeon to use the outer surface 8 to put pressure on the outside of the eye. This allows to obtain an instrument in the form of an illuminated scleral depressor, which in combination with the ophthalmic illuminator instrument 10 provides a surgeon with the advantage of e.g. absolute control of the eye during peripheral vitrectomy as it allows for visualization of the peripheral retina by depression, as shown in FIG. 4.

FIG. 4 shows a view of the combination of Illuminator end part 1 and ophthalmic illuminator instrument 10 of FIG. 1 during actual use. In FIG. 4 a cross section is shown of an eye 15, wherein the eye globe is pushed inwardly using the outer surface 8 of the light deflection part 5, thus bringing the corresponding part of the inside surface of the eye globe within the surgeon's field of view (directly through the cornea, or through a microscope. At the same time, this area is illuminated from the outside of the eye globe via the light deflection part 5. E.g. using as indicated a trocar 16 and vitrectome 17, the indenting of the eye globe and endo-illumination allows the tissue of the globe to be analysed and e.g. to perform a precise shaving action using the vitrectome 17.

The light deflection function of the Illuminator end part 1 will be explained in further detail with reference to FIG. 2, which shows a cross sectional view of the Illuminator end part 1 shown in FIG. 1, attached to an ophthalmic illuminator instrument 10 provided with a light guiding end part 11, as shown with an outer diameter d_{I}. along its entire length. In the exemplary embodiment shown, the bore 4 in the elongated sleeve part 3 tapers from an instrument adapter part side (first inner diameter d_{b}′) towards a light deflection part side (second inner diameter d_{b}). This ensures an easy entry of the light guiding end part 11 into the bore 4 of the Illuminator end part 1, and even a self-centring of the ophthalmic illuminator instrument 10 toward the light deflection elements in the light deflection part 5 in the tip of the Illuminator end part 1. The second inner diameter d_{b} near (or proximal to) the light deflection part 5 may be equal to or smaller than the outer diameter d_{I} of the light guiding end part 11. In one group of embodiments, the second inner diameter do is substantially equal to the outer diameter d_{I}, or alternatively, the second inner diameter do is smaller than the outer diameter d_{I} as the tapering inner diameter of the bore 4 still allows proper alignment of various gauge light guiding elements 11.

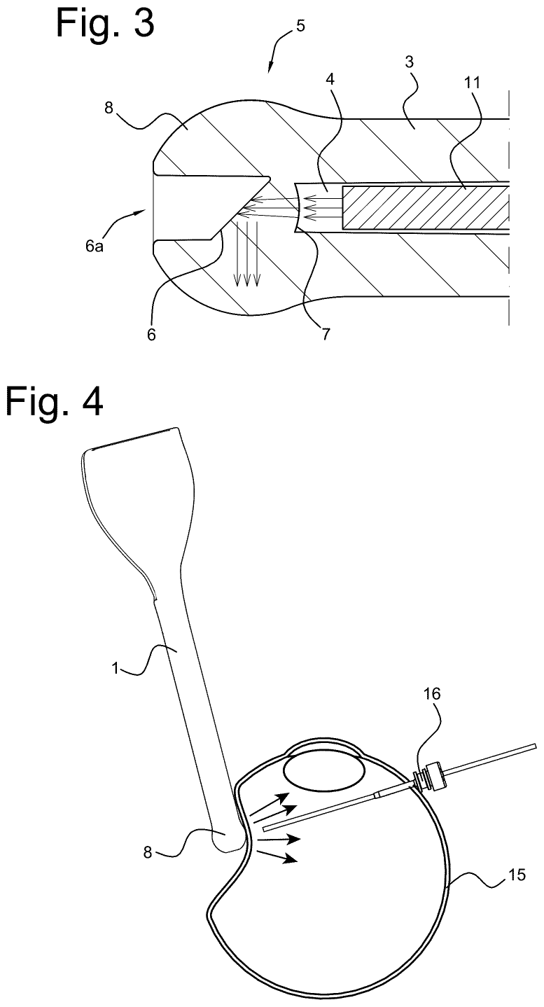

FIG. 3 shows a detailed cross sectional view of a tip of an exemplary embodiment of the Illuminator end part of FIG. 1. In this exemplary embodiment, the light deflection part 5 comprises a deflection surface 6. Light exiting from an end surface of the light guiding end part 11 is deflected away from the longitudinal direction of the Illuminator end part 1 by the deflection surface 6.

In the embodiment shown, the deflection surface 6 is at an angle of about 45 degrees to the first direction (longitudinal direction of the Illuminator end part 1). This may be implemented by providing a properly formed void 6a in the light deflection part 5 as shown in FIG. 3. The deflection surface 6 will eventually cause a diversion or deflection of the light at 90 degrees from the first direction, which is very efficient to obtain a large amount of light into an eye during use (e.g. as shown in FIG. 4). In further embodiments, the deflection surface 6 may be at a different angle, or even have an optically formed surface, e.g. to scatter light in a (limited) range of directions.

In a further embodiment, the Illuminator end part 1 further comprises a light conversion member 7 aligned in the bore 4, wherein the light conversion member 7 is arranged to focus light originating from the light guiding end part 11 onto the deflection surface 6 during operation. This would allow to obtain a larger part of the light emanating from the light guiding end part 11 to eventually exit or end up out of the light deflection part 5. E.g. the light conversion member 7 may be implemented as a concave lens surface, in more general wording, the light conversion member 7 is a focussing surface provided as part of the light deflection part 5 in a further embodiment. Such a focussing surface may be easily obtained by properly shaping a surface formed in the material of the Illuminator end part 1. In addition or alternatively, the light conversion member 7 is provided as a (separate) lens element. This would allow an even more precise definition of the light beam eventually exiting the light deflection part 5 of the Illuminator end part 1. The light conversion member 7 in the form of a lens element can also be part of the light guiding end part 11.

In FIG. 3, the arrows show the light direction in the various regions of the light deflection part 5 during operation, in this example transitioning from a parallel beam exiting the light guiding end part 11 to a converging beam between light conversion member 7 and deflection surface 6, to again a parallel beam between the deflection surface 6 and outside surface 8.

In order to properly guide light, the light deflection part 5 comprises an opaque or transparent material. In an exemplary further embodiment, the opaque or transparent material is one of a medical grade acrylonitrile butadiene styrene (ABS). poly methyl methacrylate (PMMA) or glass. E.g. ABS is sufficiently strong to allow the Illuminator end part 1 to be used as deflector, and provides a sufficiently high transparency to allow the light energy to exit from the Illuminator end part 1.

In a further embodiment, the opaque or transparent material has a refractive index higher than 1. This allows the deflection surface 6 to be formed by a material-air interface which is easy to realize using well known manufacturing techniques such as injection moulding. The Illuminator end part 1 as shown in the FIG. 1-4 embodiments can be manufactured easily using injection moulding techniques.

As shown in FIG. 4, an advantageous use of the present invention embodiments is to use the Illuminator end part 1 also for depression of (scleral) tissues. In order to safely and accurately use the Illuminator end part 1, the outer surface 8 has a continuously varying outer diameter in a further embodiment. This ensures no sharp transitions or edges are present which could damage tissue during use. In an even further embodiment, the outer surface 8 has a droplet shape, e.g. comprising a smooth convex outer surface, aiding in providing simple and effective use of the depression function of the Illuminator end part 1.

The present invention Illuminator end part 1, in a further embodiment comprises an instrument adapter part 2, allowing the illuminator end cap 1 to be attached to its associated ophthalmic instrument using the instrument adapter part 2. In an exemplary embodiment, the instrument adapter part 2 has a circumferential surface with a slit 9 in the first direction. The slit 9 in the instrument adapter part 2 is easy to manufacture (e.g. using injection moulding), and provides for a robust attachment to the associated instrument. Furthermore, the slit 9 allows a more easy introduction and alignment of the light guiding end part 11 (usually a fiber) with the bore 4, which prevents possible damage to the light guiding end part 11 while assembling. In addition, as the deflection surface 6 and slit 9 can have a fixed mutual orientation, the slit 9 may also act as a user indicator for the light emission direction of the Illuminator end part 1 (e.g. at 0 degree or at 180 degrees).

Furthermore, in a further group of embodiments (most embodiments shown in the figures and described above), the Illuminator end part 1 is a single piece component. The Illuminator end part 1 may be manufactured by injection moulding, which allows for cost-effective and material effective manufacturing, which may also allow the Illuminator end part 1 to be a single use, disposable item.

The present invention has been described above with reference to exemplary embodiments as illustrated in the drawings. The invention may also be defined as the following numbered and referenced embodiment clauses:
Embodiment 1. Illuminator end part for an ophthalmic illuminator instrument, the illuminator end part (1) comprising
an elongated sleeve part (3) having a bore (4) extending in a first direction with a diameter corresponding to an outside diameter of a light guiding end part (11) of the ophthalmic illuminator instrument (10), and
a light deflection part (5) arranged to guide light originating from the light guiding end part (11) in a direction away from the first direction.
Embodiment 2. Illuminator end part according to embodiment 1, wherein the light deflection part (5) comprises a deflection surface (6).
Embodiment 3. Illuminator end part according to embodiment 2, wherein the deflection surface (6) is at an angle of about 45 degrees to the first direction.
Embodiment 4. Illuminator end part according to embodiment 2 or 3, further comprising a light conversion member (7) aligned in the bore (4), wherein the light conversion member (7) is arranged to focus light originating from the light guiding end part (11) onto the deflection surface (6) during operation.
Embodiment 5. Illuminator end part according to embodiment 4, wherein the light conversion member (7) is a focussing surface provided as part of the light deflection part (5).
Embodiment 6. Illuminator end part according to embodiment 4, wherein the light conversion member (7) is a lens element.
Embodiment 7. Illuminator end part according to any one of embodiments 1-6, wherein the light deflection part (5) comprises an opaque or transparent material.
Embodiment 8. Illuminator end part according to embodiment 7, wherein the opaque or transparent material is medical grade acrylonitrile butadiene styrene (ABS), poly methyl methacrylate (PMMA) or glass.
Embodiment 9. Illuminator end part according to any one of embodiments 1-8, wherein the light deflection part (5) comprises an outer surface (8) for scleral depression, wherein the outer surface (8) has a maximum outer diameter larger than an outside diameter of the elongated sleeve part (3).
Embodiment 10. Illuminator end part according to embodiment 9, wherein the outer surface (8) has a continuously varying outer diameter.
Embodiment 11. Illuminator end part according to embodiment 9 or 10, wherein the outer surface (8) has a droplet shape.
Embodiment 12. Illuminator end part according to any one of embodiments 1-11, further comprising an instrument adapter part (2).
Embodiment 13. Illuminator end part according to any one of embodiments 1-12, wherein the instrument adapter part (2) has a circumferential surface with a slit (9) in the first direction.
Embodiment 14. Illuminator end part according to any one of embodiments 1-13, wherein the bore (4) in the elongated sleeve part (3) tapers from an instrument side towards a light deflection part side.
Embodiment 15. Illuminator end part according to any one of embodiments 1-14, wherein the illuminator end part (1) is a single piece component.

The present invention has been described above with reference to a number of exemplary embodiments as shown in the drawings. Modifications and alternative implementations of some parts or elements are possible, and are included in the scope of protection as defined in the appended claims.

1. An assembly of an ophthalmic illuminator instrument and an illuminator end part,
the illuminator instrument comprising
an elongate light guiding end part, extending in a first direction from an instrument side towards a light deflection part side,
the illuminator end part comprising:
an elongated sleeve part having a bore extending in a first direction with a diameter corresponding to an outside diameter of a light guiding end part of the ophthalmic illuminator instrument, and tapering from an instrument side towards a light deflection part side,
a light deflection part arranged to guide light originating from the light guiding end part in a direction away from the first direction, and
an instrument adapter part that is releasably connected to the illuminator instrument.

2. The assembly according to claim 1, wherein the light deflection part comprises a deflection surface.

3. The assembly according to claim 2, wherein the deflection surface is at an angle of about 45 degrees to the first direction.

4. The assembly according to claim 2, further comprising a light conversion member aligned in the bore, wherein the light conversion member is arranged to focus light originating from the light guiding end part onto the deflection surface during operation.

5. The assembly according to claim 4, wherein the light conversion member is a focussing surface provided as part of the light deflection part.

6. The assembly according to claim 4, wherein the light conversion member is a lens element.

7. The assembly according to claim 1, wherein the light deflection part comprises an opaque or transparent material.

8. The assembly according to claim 7, wherein the opaque or transparent material is medical grade acrylonitrile butadiene styrene (ABS), poly methyl methacrylate or glass.

9. The assembly according to claim 1, wherein the light deflection part comprises an outer surface for scleral depression, wherein the outer surface has a maximum outer diameter larger than an outside diameter of the elongated sleeve part.

10. The assembly according to claim 9, wherein the outer surface has a continuously varying outer diameter.

11. The assembly according to claim 9, wherein the outer surface has a droplet shape.

12. The assembly according to claim 1, wherein the instrument adapter part has a circumferential surface with a slit in the first direction.

13. The assembly according to claim 1, wherein the illuminator end part is a single piece component.
